(19) United States
(12) Patent Application Publication
(10) Pub. No.: US 2005/0164913 A1
(43) Pub. Date: Jul. 28, 2005

(54) Disinfecting composition

(75) Inventors: Victor S. Polyakov, Moscow (RU); Valeriy V. Ermilov, Moscow (RU); Vladimir S. Kuzmin, Moscow (RU); Oleg I. Lukashov, Moscow (RU)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Veckis Industries Ltd., Vaduz (LI)

(21) Appl. No.: 11/021,231

(22) Filed: Dec. 23, 2004

Related U.S. Application Data
(63) Continuation of application No. PCT/US03/20349, filed on Jun. 27, 2003.
Application No. PCT/US03/20349 is a continuation-in-part of application No. 10/185,024, filed on Jun. 28, 2002, now abandoned.

(51) Int. Cl. A61K 38/16; A61K 31/28

(52) U.S. Cl. 514/6; 514/492

(57) ABSTRACT

The invention relates to compositions used for disinfecting and applicable in the national economy, medicine, and laboratories of all types. The composition comprises a chelating metal complex compound comprising a monodentate, bidentate, or polydentate ligand that exhibits affinity to hydrogen ion, an ionogenic surfactant, and a solvent. The preparation displays antiseptic properties. The composition inhibits Gram positive and Gram negative bacteria, viruses, and spores. The compositions of the present invention can be applied over a broad temperature range.

### CROSS-REFERENCE RELATED APPLICATIONS

This application is a continuation application of International Application No. PCT/US03/20349, filed Jun. 27, 2003, which is a continuation in part of U.S. application Ser. No. 10/185,024, filed Jun. 28, 2002, both of which are incorporated by reference in their entirety.

### FIELD OF THE INVENTION

The present invention relates to microbiocidal or disinfectant compositions useful in the control of and/or elimination of bacteria, fungi and viruses in a wide area of applications. These compositions are useful for disinfecting and intended for use in the food industry and different sectors of the national and municipal economy, public and utility services, public catering establishments, agriculture, medicine, laboratories of all types, private life, etc. The compositions of this invention can be applied as a universal disinfecting, sterilizing, bactericidal, fungicidal and/or virucidal agents in different areas of national economy.

### BACKGROUND OF THE INVENTION

One of the well known disinfecting agents is hydrogen peroxide and preparations thereof. A representative of this group is a disinfecting preparation containing hydrogen peroxide, magnesium laurylsulphate, glycerin, sodium oleate, the disodium salt of EDTA, sodium benzoate and water (RU2108810 C1, 1998). This agent is intended for decontaminating surfaces in houses, sanitary appliances, linen, medical goods and its efficacy is not sufficient. It is not toxic to humans or animals.

Broadly known are bactericidal compositions exhibiting an increased activity containing lanthionine and a chelating agent. The suitable chelating agents are, for example, ethylenediaminotetraacetic acid (EDTA), its salts and citrate. (U.S. Pat. No. 5,260,271; U.S. Pat. No. 5,334,582)

Also known is a bactericide, which comprising a composition, including a metal complex with an α-amino acid and obtained in an acidic medium, and a disinfectant. (U.S. Pat. No. 6,242,009).

It is known that chelating metal complexes exist in an acidic medium only in negligible concentrations. (Fundamentals of Analytical Chemistra Book 1, Moscow—“Mir”—D. Skoog, D. West, 1979).

For example, a chelating agent as EDTA completely binds metal ions to form chelating complexes at pH above 6,0. For weaker chelating agents, of which natural amino acids are an example, to completely bind all metal ions into chelating complexes, the pH values of media should not be higher. The investigations carried out by the inventors have revealed that in U.S. Pat. No. 6,242,009 (the “'009 Patent”) transformation of amino acids and metal ions into nondissociating chelating complexes can occur only at pH>7.0 and addition of mineral acids in accordance with the examples cited in the patent leads to the destruction of the chelating complexes. In addition, the amino group of the amino acid is protonated and the metal exists in an ionic form. Antimicrobial activity of the compounds cited in '009 can be attributed not to the activity of chelating complexes but to metal ions, which, as is known from the literature, also exhibit certain bactericidal activity, in particular, the cited silver ions. It should be also noted that arsenic and selenium compounds are cited in the '009 as metals and their antibacterial activity can be determined by a high toxicity to all living organisms, including human. There is no doubt that the presence of strong disinfectants (chlorohexydine, hydrogen peroxide), which are introduced as additives to the complexes cited in '009 Patent, can increase the activity of the preparation.

Also described are bactericide compositions, which include cetyltrimethylammonium chloride as an active compound (DE 4326866,1995; U.S. Pat. No. 5,206,016; U.S. Pat. No. 5,575,991).

Of interest is an antiseptic preparation, which includes as an active compound cetyltrimethylammonium chloride, a mineral or an organic acid and a solvent (RU 2118174 C1). The known compound exhibits bactericidal activity towards gram negative microflora and it is not substantially effective towards intestinal and other infections of bacterial and viral etiology as well as towards anthrax.

Also known is a disinfecting preparation containing bacteriocine, a chelating agent, a stabilizer, a surfactant, a salt and an alcohol (RU 2163145). The known preparation is used for impregnating napkins which are applied for prophylaxis of mastitis in animals.

The related composition to the present invention is a disinfecting preparation which contains an active compound—a peroxide compound, a surfactant, a chelating complex and a solvent (RU20614497). This composition is active only when used at positive temperatures of 18-25° C. The prolongation of the bacteria inactivation is varied in the interval of 5-30 minutes.

### SUMMARY OF THE INVENTION

The object of the present invention is to provide a highly effective universal disinfecting, antiseptic and bactericidal, fungicidal or virucidal composition, which is useful in a broad range of positive and negative temperatures and in increasing the term of microbiocidal and disinfectant action. A further objective of the invention is to enhance the length of time of the microbiocidal or disinfectant action. The present composition is suitable for a long-term storage, is safely used, and exhibits high bactericidal, virucidal, fungicidal, and sporocidal activity and is nontoxic to animals and humans. The present antimicrobial and anti-sporicidal compositions are useful in a wide variety of utility areas. These compositions are useful as topical applications in the treatment of microbiocidal infections in a subject. Applicants' compositions can be applied to various surfaces and when so applied these compositions serve as sterilizers or sanitizers. Similarly, the present compositions can be used in application areas such as, for example, in swimming pools, spas, etc., as a laundry soap or detergent additive, as a paint or surface coating additive, as a natural or synthetic surface preservative such as the prevention of microfloral growth on surfaces such as polymers, plastics or wood, as a hard surface or carpet sanitizer. These compositions are generally useful in controlling and/or elimination of microflora and spores in many industrial, medical, agricultural, veterinary and domestic applications. Additionally, the present compositions can be employed to sterilize or disinfect gaseous environments including, for example, the cleansing of the atmosphere in homes and industrial sites, as well as airplanes, etc.

### DETAILED DESCRIPTION OF THE INVENTION

Clearly, antipathogenic compositions and methods that decrease the infectivity, morbidity, and mortality associated with pathogenic exposure are needed. Such compositions and methods should preferably not have the undesirable properties of promoting microbial resistance, or of being toxic to the recipient.

The present invention relates to compositions and methods for decreasing the infectivity, morbidity, and rate of mortality associated with a variety of pathogens. The present invention also relates to method and compositions for decontaminating areas, samples, surfaces solutions, and foodstuffs colonized or otherwise infected by pathogens and microorganisms.

In some embodiments, the present invention provides compositions and methods suitable for treating animals, including humans, exposed to pathogens or the threat of pathogens. In some embodiments, the animal is contacted with effective amounts of the compositions prior to exposure to pathogenic organisms. In other embodiments, the animal is contacted with effective amounts of the compositions after exposure to pathogenic organisms. Thus, the present invention contemplates both the prevention and treatment of microbiological infections.

In other embodiments, the present invention provides compositions and methods suitable for decontaminating areas, solutions and surfaces, including organic and inorganic samples that are exposed to pathogens or suspected of containing pathogens. In still other embodiments of the present invention, the compositions are used as additives to prevent the growth of harmful or undesired microorganisms in biological and environmental samples.

As used herein the term “microorganism” refers to microscopic organisms and taxonomically related macroscopic organisms within the categories of algae, bacteria, fungi (including lichens), protozoa, viruses, and subviral agents. The term microorganisms encompasses both those organisms that are in and of themselves pathogenic to another organism (e.g., animals, including humans, and plants) and those organisms that produce agents that are pathogenic to another organism, while the organism itself is not directly pathogenic or infective to other organisms. As used herein the term “pathogen,” and grammatical equivalents, refers to an organism, including microorganisms, that causes disease in another organism (e.g., animals and plants) by directly infecting the other organism, or by producing agents that causes disease in another organism (e.g., bacteria that produce pathogenic toxins and the like).

The terms “host” or “subject,” as used herein, refer to organisms to be treated by the compositions' present invention. Such organisms include organisms that are exposed to, or suspected of being exposed to, one or more pathogens. Such organisms also include organisms to be treated so as to prevent undesired exposure to pathogens. Organisms include, but are not limited to animals (e.g., humans, domesticated animal species, wild animals) and plants.

As used herein, the term “inactivating,” and grammatical equivalents, means having the ability to kill, eliminate or reduce the capacity of a pathogen to infect and/or cause a pathological response in a host.

As used herein, the terms “contacted” and “exposed,” refers to bringing one or more of the compositions of the present invention into contact with a pathogen or a sample to be protected against pathogens such that the compositions of the present invention may inactivate the microorganism or pathogenic agents, if present. The present invention may inactivate the microorganism or pathogenic agents, if present. The present invention contemplates that the disclosed compositions are contacted to the pathogens or microbial agents in sufficient volumes and/or concentrations to inactivate the pathogens or microbial agents.

As used herein the term “topically active agents” refers to compositions of the present invention that elicit pharmacological responses at the site of application (contact) to a host.

As used herein the term “surface” is used in its broadest sense. In one sense, the term refers to the outermost boundaries of an organism or inanimate object (e.g., vehicles, buildings, and food processing equipment, etc.) that are capable of being contacted by the compositions of the present invention (e.g., for animals: the skin, hair, and fur, etc., and for plants: the leaves, stems, flowering parts, and fruiting bodies, etc.). In another sense, the term also refers to the inner membranes and surfaces of animals and plants (e.g., for animals: the digestive tract, vascular tissues, and the like, and for plants: the vascular tissues, etc.) capable of being contacted by compositions by any of a number of transdermal delivery routes (e.g., injection, ingestion, transdermal delivery, inhalation, and the like).

In specific embodiments, the contacting is performed for a time sufficient to kill the pathogenic agent or to inhibit the growth of the agent. In other embodiments, the present invention provides a method of decontaminating an environmental surface or area or atmosphere harboring harmful or undesired pathogens. In one such embodiment, the pathogenic agent is associated with an environmental surface and the method comprises contacting the environmental surface with an amount of the composition sufficient for decontaminating the surface. While it may be so desired, decontamination need not result in total elimination of the pathogen. In some embodiments, the compositions and methods may further comprise dyes, paints, and other marking and identification compounds so as to ensure that a treated surface has been sufficiently treated with the compositions of the present invention.

When the present compositions are administered as topical pharmaceuticals, it is contemplated that the compositions further comprise pharmaceutically acceptable adjutants, excipients, stabilizers, diluents, and the like. In still further embodiments, the present invention contemplates compositions further comprising additional pharmaceutically acceptable bioactive molecules. In the case of pharmaceutical activity, the effective amount relates to the dosage useful in achieving the desired end result. Such dosages are dependent upon the subject, i.e., age and size, etc. and can be easily ascertained by those skilled in this art.

Elimination of pathogenic micro-organisms on various surfaces, especially hard surfaces where such organisms may stay active for relatively long periods of time, has long been a goal of those charged with cleaning and maintaining an antiseptic kitchen and bathrooms in the home, as well as in commercial and institutional settings such as hospitals, medical clinics, hotels and restaurants. A further goal has been to prevent the formation of allergens caused by growth of mold and mildew on bathroom surfaces.

This invention further relates to cleaning, sanitizing, disinfecting and mold and mildew inhibiting compositions for non-porous hard surfaces such as glass (e.g., mirrors and shower doors), glazed porcelain, metallic (e.g. chrome, stainless steel, and aluminum), ceramic tile, enamel, fiberglass, Formica®, Corian®, and plastic.

When used in antiseptic applications, the methods and compositions of the invention can be used to treat a broad spectrum of infections by pathogenic microbes, preferably with a minimum of damage to normal flora. As used herein, “pathogenic microbes or microorganisms” is intended to include pathogenic bacteria, fungi, etc. which do not normally reside in the host or which have over-populated in the host to a pathogenic degree. Microbes or microorganisms which result in pathogenic infection of a host are well known. Thus, the methods and compositions of the invention can be used in the treatment of prophylaxis of infection by pathogenic microbes associated with any condition permitting delivery of the compositions of the invention to the site of infection the site of infection, including, without limitation, the treatment of superficial or surgical wounds, burns or other significant epidermal damage such as toxic epidermal necrolysis, urinary tract infections such as cystitis and urethritis, vaginitis such as vulvovaginitis and cervicitis, gingivitis, otitis externa, acne, external fungal infections, upper respiratory tract infections, gastrointestinal tract infections, subacute bacterial endocarditis and other bacterial or fungal infections to which the compositions of the invention can be effectively delivered. Pathogenic microbes which can be selectively killed in the practice of the invention include, without limitation, Streptococcus pyogenes, Streptococcus agalactiae, Staphylococcus aureus, S. pneumoniae, E. faecalis, S. epidermidis, Pseudomonas aeurginosa, Escherichia coli, Bacillis substilis and other coliformn bacteria, Candida albicans and T. rubrum and other infectious bacteria fungi.

The antiseptic compositions can be administered in any effective pharmaceutically acceptable form to warm blooded animals, including humans and animal subjects, e.g., in topical dosage forms, such as a topical, buccal, or nasal spray or in any other manner effective to deliver to a site of microbe infection. The route of administration will preferably be designed to obtain direct contact of the antiseptic compositions with the infecting microbes.

For topical applications, the pharmaceutically acceptable carrier may take the form of liquids, creams, lotions, or gels, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration. Compositions of the invention may be impregnated into absorptive materials, such as sutures, bandages, and gauze, or coated on to the surface of solid phase materials, such as staples, zippers and catheters to deliver the compositions to a site of microbe infection. Other delivery systems of this type will be readily apparent to those skilled in the art.

For topical applications, the pharmaceutically acceptable carrier may take the form of a liquid, cream, foam, lotion, or gel, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration.

The present invention also contemplates that certain compositions described herein may be employed in the food processing and preparation industries in preventing and treating food contaminated with food borne bacteria, fungi and toxins. Thus, such compositions may be employed to reduce or inhibit microbial growth or otherwise abrogate the deleterious effects of microbial contamination of food. For these applications, the present compositions are applied in food industry acceptable forms such as additives, preservatives or seasonings.

For such applications, acceptable carriers may take the form of liquids, creams, foams, gels and may additionally comprise solvents, emulsifiers, gelling agents, moisturizers, stabilizers, wetting agents, preservatives, sequestering agents, dyes, perfumes and other components commonly employed in the food processing industry.

In another embodiment of the present invention, the food compositions may be specifically designed for applications such as disinfecting or sterilization of food industry devices, equipment and areas where food is processed, packaged and stored. For applications of this type, the compositions may be conveniently provided in the form of a liquid or foam, and may be provided with emulsifiers, surfactants, buffering agents, wetting agents, preservatives, and other components commonly found in compositions of this type. In some embodiments, the compositions are applied to produce or agricultural products prior to or during transportation of those goods. Compositions of the invention may be impregnated into absorptive materials commonly used in packaging material for the prevention of food contamination during transport and storage (e.g., cardboard or paper packaging). Other delivery systems of this type will be readily apparent to those skilled in the art.

In general, the present invention contemplates compositions and methods that find use as environmental decontamination agents and for treatment of casualties in both military and terrorist attack. The inactivation of a broad range of pathogens, including vegetative bacteria and enveloped viruses and bacterial spores, combined with low toxicity in experimental animals, makes the present compositions suitable for use as general decontamination agents before a specific pathogen is identified. Preferred compositions of the present invention can be rapidly produced in large quantities and are stable for many months at a broad range of temperatures. These properties provide a flexibility that is useful for a broad range of decontamination applications.

For example, certain formulations of the present invention are effective at destroying many of the bacterial spores and agents used in biological warfare. In this regard, the compositions and methods of the present invention are useful in decontaminating personnel and materials contaminated by biological warfare agents. Solutions of present compositions may be sprayed directly onto contaminated materials or personnel from ground based, or aerial spraying systems. In certain of these applications, the present invention contemplates that an effective amount of composition be contacted to contaminated materials or personnel such that decontamination occurs. Alternatively, personal decontamination kits can be supplied to military or civilians likely to become contaminated with biological agents.

The inactivation of a broad range of pathogens, including vegetative bacteria and enveloped viruses combined with low toxicity makes the present compositions particularly well suited for use as general decontamination agents before a specific pathogen is identified.

Thus, certain embodiments of the present invention specifically contemplate the use of the present compositions in disinfectants and detergents to decontaminate soil, machinery, vehicles and other equipment, and waterways that may have been subject to an undesired pathogen. Such decontamination procedure may involve simple application of the formulation in the form of a liquid spray or may require a more rigorous regimen. Also, the present compositions can be used to treat crops for various plant viruses (in place of or for use with conventional antibiotics). The instant compositions may also be used to decontaminate farm animals, animal pens, surrounding surfaces, and animal carcasses to eliminate, for example, nonenveloped virus of hoof and mouth disease.

In addition to their use in decontamination of land and equipment, the formulations also find use in household detergents for general disinfectant purposes. Moreover, some embodiments of the present invention can be used to prevent contamination of food with bacteria or fungi (e.g., non-toxic compositions). This can be done either in the food preparation process, or by addition to the food as an additive, disinfectant, or preservative.

The inventive compositions can be used on hard surfaces in liquid or aerosol form. Accordingly, the foregoing components are admixed with one or more suitable aqueous or non-aqueous carrier liquids. The choice of carrier is not critical. However, it should be safe and it should be chemically compatible with the inventive compositions. In some embodiments, the carrier liquid may comprise solvents commonly used in hard surface cleaning compositions. Such solvents should be compatible with the inventive compositions and should be chemically stable at the pH of the present compositions. Solvents for use in hard surface cleaners are described, for example, in U.S. Pat. No. 5,108,660, herein incorporated by reference in its entirety.

The present invention further relates to decontaminating a sample by treating the sample with the instant antimicrobial compositions such that bacteria, virus, fungi or spores on the surface are killed or disabled. The surfaces contemplated may be solid surfaces such as the surfaces in homes or industrial facilities or medical facilities or the surfaces of medical devices. Additionally the surface may be the surface of an organism and can be an internal or external organism surface. The surface further can be the surface of a food product.

The present compositions can be sprayed into an atmosphere to inactivate harmful microorganisms in the atmosphere. Such spray disinfectants are readily formulated by the skilled artisan and the choice of carrier is within the skill in the art.

The present invention further relates to compositions and method for decreasing the infectivity, morbidity, and rate of mortality associated with a variety of pathogens, as well as to method and compositions for decontaminating areas, samples, solutions, and foodstuffs colonized or otherwise infected by pathogens and microorganisms.

The present invention comprises a microbiocidal or antisporicidal containing an ionogenic surfactant, a chelating complex and a solvent. According to the invention, the chelating complex comprises a metal compound, containing a monodentate, bidentate or polydentate ligand, which exhibits affinity towards the hydrogen ion, and together with the surfactant is in the proportion of about 1 to about (7-9) to the solvent.

The chelating metal complex compound containing the ligand of this invention is a chelating complex compound with a metal such as copper, zinc, mercury, chromium, manganese, nickel, cadmium, arsenic, cobalt, aluminum, lead, selenium, platinum, gold, titanium or tin or combinations thereof.

The bi- and polydentate ligands are, for example, anions of natural amino acids, iminodiacetic or nitriletriacetitic acids as well as carbon-substituted (in the a-position to the carboxylic group) derivatives of iminodiacetic and nitriletriacetic acids with various residues of amino acids fragments containing no aminocarboxylic group, alkylenediaminopolyacetic acid, as well as carbon-substituted (in the α-position to the carboxylic group) derivatives of polyalkylenepolyaminopolyacetitc acids with various residues of aminoacetic fragments containing no aminocarboxylic group, derivatives of ω-phosphoncarboxylic and ethylenediphosphontetrapropionic acids, derivatives of ethelynetetra(thioacetic) and diethylenetrithiodiacetic acids, monoamine complexones, in which carboxylic groups are replaced by phosphonic groups, or mixtures thereof.

The chelating metal complex compound containing a monodentate, bidendalex or polytentate ligand can be a chelating complex compound with at least one amino acid such as for example isoleucine, phenylalanine, leucine, lysine, methionine, threonine, tryptophan, valine, alanine, glycine, arginine, histidine, or mixtures thereof.

An embodiment of the invention comprises a microbiocidal or sporicidol composition containing an ionogenic surfactant, a chelating complex and a solvent, wherein the chelating complex comprises a chelating metal complex compound containing a monodentate, bidentate or polydentate, ligand, which exhibits affinity to hydrogen ion, and the solvent comprises a mixture of water and an aliphatic alcohol (C₁-C₈) with the following ratio, weight %:

| | |
|---|---|
| Chelating complex metal compound, containing a | about 1-30 |
| monodentate, bidentate or polydendate ligand which | |
| exhibits affinity to hydrogen ion | |
| Ionogenic surfactant | about 0.1-15 |
| Aliphatic alcohol (C₁-C₈) | about 0.5-95 |
| Distilled water | Remainder |

An aspect of the invention comprises a disinfecting composition comprising an ionic surfactant, a chelating complex and a solvent. The chelating complex comprises a chelating complex metal compound, which includes, along with commonly used mono-, bi- and polydentate ligand, an additional monodentate ligand exhibiting affinity towards hydrogen ion, and exemplary solvents, include distilled water and an aliphatic alcohol (C₁-C₈) with the following weight % ratio:

| | |
|---|---|
| Chelating metal complex compound, containing a | about 1-30 |
| monodentate, bidentate or polydentate ligand and | |
| exhibiting affinity towards hydrogen ion | |
| Ionogenic surfactant | about 0.1-15 |
| Aliphatic alcohol (C₁-C₈) | about 0.5-95 |
| Distilled water | remainder |

Exemplary chelating metal complex compounds comprise glycinatecopper chloride complex and the ethylenediaminotetraacetate zinc complex.

Suitable halogen containing ionogenic compounds may be selected, for example, from compounds comprising chloride, fluoride, bromide and iodide ions. In preferred embodiments, suitable cationic halogen containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridiniumj chloride (CPC), cetyltrimethylammonium chloride, cetylbenzyldimethylammonium chloride, cetylpyridinium bromide (CPB), cetyltrimethylammkonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

Exemplary ionogenic surfactants comprise cetylpyridinium halogenides and cetyltrimethylammonium halogenides.

Metal complex compounds are useful disinfecting and antibacterial preparations. They are bactericidal reagents exhibiting a broad range of antibacterial action, irreversibly killing a pathogenic microflora. The mechanism of action of metal complex compounds is based on blocking amino acid groups of a protein shell and enzyme systems of microorganisms. At the first stage there are formed associates with a chelating complex and then a monodentate, bidentate or polydentate ligand is substituted by an amino acid group of protein, which leads to a complete blocking of metabolic processes in microorganisms and to their death.

By the toxic action on a human organism, the proposed compounds relate to the IY class of danger. Doses, which are used in practice for antibacterial treatment, do not cause a pronounced toxic or irritating effect on skin or mucosa.

The proposed compositions based on chelating metal complex compounds do not exert on influence animal or human organisms because the compounds containing amino acid groupings are withdrawn from the organism by the exchange reaction. Bactericidal chelating complexes practically do not affect the most important living functions of the organism.

The proposed bactericides relate to metal complexes with chelating ligands, which are obtained in the alkaline and not in the acidic pH range. Therefore, the proposed compositions compared to the analogs have a broader field of application because they are ecologically safe and possess low toxic and hygienic characteristics based on a different mechanism of bactericide action. In addition, the proposed compositions exhibit an increased chemical stability towards environmental impact (stability constants of the proposed complexes are several orders higher than those of the closest analogs).

Useful monodentate bidentate or polydentate ligands include ligands exhibiting affinity towards hydrogen ion, which determines their ability to be substituted by an amino group of protein in a microorganism.

A molecule of the proposed bactericide contains a metal ion preferably, for example, copper (II) and zinc, as well as monodentate bidentate or polydentate ligands, exhibiting affinity towards hydrogen ion, such as ammonia, mono-, di- and triethanolamines and others.

The pH of the obtained bactericidal compositions is about ≧7.0.

For the synthesis of bactericides, use is made of metal salts. The synthesis is carried out in aqueous solutions by stirring the ingredients at room temperature. The monodentate ligands used are water soluble substances which display affinity towards a hydrogen ion.

The distinguishing characteristic of the present bactericide compositions is that the interaction (mixing) of the ingredients takes place in neutral and alkali media at pH≧about 7.0 in the absence of mineral acids.

As for the parameters of the disinfecting activity, it is established that the present microbiocidal and sporicidal compositions are sufficient and do not require the use of any additional disinfecting preparations, for example, chlorohexydine, hydrogen peroxide, etc.

The method for synthesis of the glycinatecopper chloride complex and ethylenediaminotetraacetate zinc complex is known from the following sources:
Ley, Berichte, V. 42, S. 371;
Hofmeister, “Beittage zur Kenntiniss der Amidosäurcn” Annalen der Chemie, 1877 V.189, S.36
“Synthetic Production and Utilization of Amino Acids”, Ed. T. Kaneko, Y. Izumi, I. Chibata, Wiley, N.-Y., 1974.
Dyatlova N. M. et al., Complexones and Metal Complexonates, M.: —<<Khimiya>> 1988.

The antimicrobial activity of the glycinatecopper chloride complex, ethylenediaminotetraacetate zinc complex and compositions thereof was investigated in the Scientific Research Disinfectology Institute, Moscow (the data are given in the report of the Institute of 15.02.2002).

The ingredients ratio in the proposed compositions is selected so as to provide for optimal technological characteristics of the preparation and for retaining the stable properties.

The concentrations ranges in the compositions:

| | |
|---|---|
| Chelating metal complex | about 1%-30% |
| Ionogenic Surfactant (quaternary ammonium | about 0.1%-15% |
| halogenides-, C₁₂-C₁₆-alkyltrimethylammonium, | |
| di(C₈-C₁₀-alkyl)dimethylammonium, | |
| in particular cetylpyridinium and | |
| cetyltrimethylammonium halogenides | |
| Aliphatic alcohol (C₁-C₈) | about 0.5%-95% |
| Water | about 3%-98% |

The proposed concentrations ranges for the ingredients in the composition are determined by the object to achieve the above mentioned bactericidal, fungicidal and sporocidal efficiency of the composition.

The technical result is possible to achieve by making use of—as ionogenic surfactants—quaternary ammonium halogenides, in particular C₁₂- C₁₆ alkyltrimethylammonium, di(C₈-C₁₀-alkyl)dimethylammonium, C₁₂- C₁₆-alkylpyridinium, in particular cetylpyridinium and cetyltrimethylammonium halogenides.

Industrial application of the proposed preparation is confirmed by the following examples.

### EXAMPLE 1

2.0 g of sodium hydroxide is dissolved in 50 cm³ of distilled water in a flask and 3.75 g of glycine is added on stirring. 6.8 g of zinc chloride is added portion-wise to the obtained solution on stirring followed by the addition of 3.75 cm³ of 25% aqueous solution of ammonium. Separately there is prepared a solution of 0.43 g of cetyltriethylammonium chloride in the mixture of 1.2 g of tryethyleneglycol and 15.3 cm³ of water. Both solutions are mixed and diluted with water to achieve the concentration which is required for the antibacterial treatment of objects.

### EXAMPLE 2

To 6.1 cm³ of 25% solution of ammonia in a flask there are added 25 ml of water and 11.85 g of ethylenediaminotetraacetic acid. On stirring, there is added portion-wise 5.45 g of copper dichloride and 2.4 g of ethanolamine is poured. The formed solution turns dark blue. Separately there is prepared a solution of 8.1 g dodecylbenzyltrimethylammonium chloride in a mixture of 7.3 cm³ of isopropyl alcohol and 10 cm³ of water. Both solutions are mixed and diluted to achieve the concentration required for the antibacterial treatment of objects.

### EXAMPLE 3

In flask 0.4 g of sodium hydroxide is dissolved in 20 cm³ of distilled water and 1.46 g of L-lysine is added on stirring. Then 1.36 g of zinc chloride is added portion-wise on stirring. The obtained solution is mixed with 0.75 cm³ of 25% solution of ammonium in water. Separately there is prepared a solution of 12.0 g of cetylpyridinium chloride in 56.0 cm³ of isopropyl alcohol. An aqueous solution of a zinc amino acid complex is added slowly, portion-wise. The mixture is stirred and diluted with water to achieve the concentration, which is required for the antibacterial treatment of objects.

### EXAMPLE 4

A chelating metal complex compound containing a monodentate ligand, which displays affinity towards hydrogen ion, is mixed with an ionogenic surfactant, in particular as is indicated in Example 1. Distilled water is added to achieve the 10% or 30% concentration, i.e., the ratio with the solvent of 1-9 or 7.

### EXAMPLE 5

The ingredients are mixed as is described in Example 2 in the following amounts (%):

| | |
|---|---|
| Chelating metal complex compound containing | −30 |
| a monodentate, bidentate or polydentate ligand, | |
| which displays affinity towards hydrogen ion | |
| Ionogenic surfactant | −15 |
| Aliphatic alcohol (C₁-C₈) | −0.5 |
| Distilled water | −54.5 |

### EXAMPLE 6

The ingredients are mixed as is described in Example 2, in weight %:

| | |
|---|---|
| Chelating metal complex compound containing | 2 |
| a monodentate, bidentate or polydentate ligand, | |
| which displays affinity towards hydrogen ion | |
| Ionic surfactant | −1 |
| Aliphatic alcohol (C₁-C₈) | −95 |
| Distilled water | −2 |

### EXAMPLE 7

The ingredients are mixed as is described in Example 3 in the following mass %:

| | |
|---|---|
| Chelating metal complex compound containing | −1 |
| a monodentate ligand, which displays affinity | |
| towards hydrogen ion | |
| Ionogenic surfactant | −5 |
| Aliphatic alcohol (C₁-C₈) | −20 |
| Distilled water | −74 |

### EXAMPLE 8

The ingredients are mixed as is described in Example 3 in the following mass %:

| | |
|---|---|
| Chelating metal complex compound containing | −2 |
| a monodentate, bidentate or polydentate ligand | |
| which displays affinity towards hydrogen ion | |
| Ionogenic surfactant | −0.1 |
| Aliphatic alcohol (C₁-C₈) | −30 |
| Distilled water | −67.9 |

Bactericidal Activity

To investigate the disinfecting properties of samples, as test microorganisms, use was made of vegetative forms of bacteria E. coli (strain 1257), which simulates pathogenes of intestinal infections—gram negative bacteria; Staphylococcus aureus (strain 906), which simulates infections of respiratory tract and is a pathogen of hospital infections—gram positive bacteria, as well as of bacteria Bacillus cereus (strain 96), which simulates an anaerobic infection—gas gangrene, tetanus and anthrax.

The initial investigations of chelating metal complexes, for example of glycinatecopper ammonium chloride, have revealed their high enough efficacy towards the vegetative forms (see Table 1).

TABLE 1
Antimicrobial activity of samples

| | | | Death time of | | |
|---|---|---|---|---|---|
| | | Concen- | test-microorganisms (min) | | |
| No | | tration | | Staphylococcus | Bacillus |
| π/π | Sample | (%) | E. coli, | aureus, | cereus. |
| 1 | Ethylenediamino- | 0.1 | >30 | >30 | — |
| | tetraacetate zinc | 5.0 | >30 | >30 | |
| | complex | | | | |
| 2 | Monoglycinatecopper | 0.1 | >30 | >30 | — |
| | chloride | 0.2 | 30 | >30 | — |
| | complex | 0.5 | 15 | >30 | — |
| | | 5.0 | | >30 | >360 |
| 3 | Preparation 1 on | 0.025 | 30 | >30 | — |
| | the basis of | 0.05 | 5 | 5 | |
| | glycinatecopper | 2.0 | 5 | 5 | <60 |
| | chloride complex | | | | |
| 4 | Preparation 2 on | 0.05 | 5 | 5 | — |
| | the basis of | 0.1 | 5 | 5 | — |
| | ethylenediamino- | 5.0 | 5 | 5 | <60 |
| | tetraacetate zinc | | | | |
| | complex | | | | |

For increasing antibacterial efficacy, in particular sporocidal properties, ionogenic surfactants (cetylpyridinium chloride, cetyltrimethylammonium bromide) were introduced into the solutions of chelating metal complex compounds. Thus the obtained composition on the basis of glycinatecopper ammonium chloride and cetyltrimethylammonium bromide (preparation 1, see Table 1) displays the synergism of action towards gram negative and gram positive bacteria.

Among the preparations on the basis of chelating zinc complexes, the highest activity towards the aforementioned types of bacteria is displayed by preparation 2 (see Table 1), which is based on 2-aminoethanol ethylenediaminotetraacetate zinc complex and cetylpyridinium chloride.

The investigations of the preparation, which consists of 5% solution of ethylenediaminotetraacetate zinc in a water-alcohol solution (70 vol. % isopropyl alcohol), have revealed activity towards vegetative types of bacteria on a 128-fold dilution, while towards anthrax (spores)—on a 16-fold dilution.

The proposed universal ecologically safe bactericidal preparation is intended for disinfecting the main forms and types of pathogenic microflora including the spore form. The preparation exhibits increased ecological properties, which is achieved by applying nontoxic chelating agents transforming metal ions into nontoxic chelating complexes.

The preparation makes it possible:
To reduce the cost of the bactericide complex;
To increase environmental stability due to the fact that the proposed bactericide chelating metal complexes are independent on such environmental factors as temperature, humidity, light effect;
To retain operating properties for many years.

In the present investigations, there is established that bactericidal effect and stability of the preparation are decreased in case the ingredients content is lower than the pointed minimal values of the composition.

The present standard investigations have revealed high efficiency of the preparation towards such pathogens as:
Intestinal infections (gram negative bacteria)—pseudomonas aeruginosa, dysenteria, salmonellosis
Respiratory tract and hospital infections (gram positive bacteria)—staphylococcosis, streptococcosis, microflora et al.;
Anaerobic infections—wound infections (tetanus)
Anthrax (spores) et al.

The preparation effectively acts on viruses (hepatitis, herpes, AIDS-infection, rotaviral infections).

Buffering of the bactericide composition provides for the desirable bactericidal effect at all pH values of a human skin, the pH value of the preparation is weakly alkalin, i.e., about 7.6±0.5.

The area of application of the preparation is that of prophylaxis and disinfecting of contaminated open parts of human and animal skin as well as of surfaces of the majority of materials.

By its content and principal of action, the preparation is safe for humans and animals, nontoxic, does not irritate skin, chemically neutral towards all construction materials and fabrics based on natural and synthetic fibers, does not cause corrosion of metals.

If the composition is applied over skin, hair, nail and mucous membrane, the bactericide effect is retained for not less than 2 hours; while applied over surfaces of materials, fabrics, and protective coverings—24 hours and above.

The temperature range for skin application is from about −20° C. to about +40° C. to about +50° C.; for surfaces about −50° C. to about +50° C. The preparation kills 99.99% of microbes.

By acute toxicity, the preparation is related to the IY class of low hazard compounds.

A mixture of effective amounts of ingredients exhibits a synergetic effect and disinfecting properties are increased.

All references cited in this application are expressly incorporated herein by reference hereto.

It will be understood by those skilled in the art that various modifications and substitutions may be made to the invention as described above without departing from the spirit and scope of the invention. Accordingly, it is understood that the present invention has been described by way of illustration and not limitation.

1. A microbiocidal disinfectant composition comprising an ionogenic surfactant, a chelating complex and a solvent, wherein the chelating complex comprises a chelating metal complex compound containing a monodentate ligand, bidentate or polydentate ligand which exhibits affinity to hydrogen ion, and wherein the ratio of the ligand and ionogenic surfactant to the solvent being about 1 to about (7-9).

2. The disinfectant composition according to claim 1, wherein the chelating metal complex compound containing the ligand is chelating complex compound with a metal selected from copper, zinc, mercury, chromium, manganese, nickel, cadmium, arsenic, cobalt, aluminum, lead, selenium, platinum, gold, titanium, tin or combinations thereof.

3. The disinfecting composition according to claim 1, wherein the mono-, bi- and polydentate ligands are selected from anions of natural amino acids, iminodiacetic or nitriletriacetitic acids as well as carbon-substituted (in the α-position to the carboxylic group) derivatives of iminodiacetic and nitriletriacetic acids with various remnants of amino acids fragments containing no aminocarboxylic group, alkylenediaminopolyacetic acid, as well as carbon-substituted (in the α-position to the carboxylic group) derivatives of polyalkylenepolyaminopolyacetitc acids with various remnants of aminoacetic fragments containing no aminocarboxylic group, derivatives of ω-phosphoncarboxylic and ethylenediphosphontetrapropionic acids, derivatives of ethelynetetra(thioacetic) and diethylenetrithiodiacetic acids, monoamine complexones, in which carboxylic groups are replaced by phosphonic groups, or mixtures thereof.

4. The disinfecting composition according to claim 1, wherein the chelating metal complex compound containing a monodentate bidentate or polydentate ligand is a chelating complex compound with at least one amino acid selected from isoleucine, phenylalanine, leucine, lysine, methionine, threonine, tryptophan, valine, alanine, glycine, arginine, histidine, or mixtures thereof.

5. The composition of claim 1, wherein the solvent is water, preferably distilled water.

6. A disinfectant composition comprising an ionogenic surfactant, a chelating complex and a solvent, wherein the chelating complex comprises a chelating metal complex compound containing a monodentate, bidentate or polydentate, ligand, which exhibits affinity to hydrogen ion, and the solvent comprises a mixture of water and an aliphatic alcohol (C₁-C₈ ) with the following ratio, weight %:

| | |
|---|---|
| Chelating complex metal compound, containing | about 1-30 |
| a monodentate, bidentate or polydentate ligand | |
| which exhibits affinity to hydrogen ion | |
| Ionogenic surfactant | about 0.1-15 |
| Aliphatic alcohol (C₁-C₈) | about 0.5-95 |
| Distilled water | the remainder |

7. The disinfectant composition, according to claim 1, wherein the chelating complex metal compound is a glycinatecopper halide complex.

8. The disinfectant composition, according to claim 1, wherein the chelating complex compound of a metal is a ethylenediaminotetraacetate zinc complex.

9. The disinfectant composition, according to claim 1, wherein the ionogenic surfactant is a cetylpyridinium halogenide.

10. The disinfectant composition, according to claim 1, wherein the ionogenic surfactant is a cetyltrimethylammonium halogenide.

11. The disinfectant composition according to claim 6, wherein the chelating metal complex compound containing the ligand is chelating complex compound with a metal selected from copper, zinc, mercury, chromium, manganese, nickel, cadmium, arsenic, cobalt, aluminum, lead, selenium, platinum, gold, titanium, tin or combinations thereof.

12. The disinfecting composition according to claim 6, wherein the mono-, bi- and polydentate ligands are selected from anions of natural amino acids, iminodiacetic or nitriletriacetitic acids as well as carbon-substituted (in the α-position to the carboxylic group) derivatives of iminodiacetic and nitriletriacetic acids with various remnants of amino acids fragments containing no aminocarboxylic group, alkylenediaminopolyacetic acid, as well as carbon-substituted (in the α-position to the carboxylic group) derivatives of polyalkylenepolyaminopolyacetitc acids with various remnants of aminoacetic fragments containing no aminocarboxylic group, derivatives of ω-phosphoncarboxylic and ethylenediphosphontetrapropionic acids, derivatives of ethelynetetra(thioacetic) and diethylenetrithiodiacetic acids, monoamine complexones, in which carboxylic groups are replaced by phosphonic groups, or mixtures thereof.

13. The disinfecting composition according to claim 6, wherein the chelating metal complex compound containing a monodentate, bidentate or polydentate ligand is a chelating complex compound with at least one amino acid selected from isoleucine, phenylalanine, leucine, lysine, methionine, threonine, tryptophan, valine, alanine, glycine, arginine, histidine, or mixtures thereof.

14. The disinfecting composition, according to claim 6 wherein the chelating complex metal compound containing a monodentate, bidentate or polydentate ligand or glycinatecopper chloride complex.

15. The disinfecting composition, according to claim 6 wherein the chelating complex compound of a metal or ethylenediaminotetraacetate zinc complex.

16. The disinfecting composition, according to claim 6, wherein the ionogenic surfactant, a cetylpyridinium halogenide.

17. The disinfecting composition, according to claim 6, wherein the ionogenic surfactant is a cetyltrimethylammonium halogenide.

18. A topical microbiocidal composition comprising the microbiocidal disinfectant composition according to claim 1 and a suitable carrier therefore.

19. A composition suitable for cleaning and sanitizing a surface or atmospheric area comprising the microbiocidal disinfectant composition of claim 1 and optionally a suitable carrier.

20. The composition of claim 19, wherein the surface is a non-porous hard surface.

21. The composition of claim 20, wherein the surface or atmospheric area is in a household, commercial or institutional setting.

22. A method of decontaminating a sample comprising treating the sample with the antimicrobial disinfectant composition of claim 1 under conditions such that said composition in physical contact with the sample kills bacteria, virus, fungi or spores on said sample or disables bacteria, virus, fungi or spores on said sample.

23. The microbiocidal disinfectant composition of claim 1 formulated to coating substrates such as wood, metal, textile, thread, canvas, carpeting, paper, cement, concrete or masonry.

24. A method of sanitizing water in swimming pools, spas, and hot tubs whereby the level of pathogenic organisms in the water is lowered comprising treating the water with an effective amount of the microbiocidal disinfectant composition of claim 1.

25. A protective coating composition useful in providing a protective layer for substrate on which the coating is applied wherein said protective coating provides a microbiocidal or disinfectant effect, said composition comprises an effective amount of the microobiocidal composition of claim 1 and a suitable vehicle therefor.
